(19) United States
(12) Patent Application Publication
(10) Pub. No.: US 2012/0135055 A1
(43) Pub. Date: May 31, 2012

(54) Dry Powder Inhaler Formulations

(75) Inventors: Ian Cameron Gardner Mcaffer, Kent (GB); Peter Ernest Tasko, Stevenage (GB); Susheela Ginafrancesco, Stevenage (GB); Graham John Swift, Stevenage (GB)

(73) Assignee: BREATH LIMITED, Stevenage (GB)

(21) Appl. No.: 13/390,286

(22) PCT Filed: Aug. 16, 2010
(86) PCT No.: PCT/EP2010/061915

§ 371 Date: Feb. 13, 2012

(30) Foreign Application Priority Data
Aug. 14, 2009 (GB) 0914231.6

(51) Int. Cl. A61K 9/14; A61P 11/06; A61P 11/00; A61K 31/573

(52) U.S. Cl. 424/400; 514/180

(57) ABSTRACT

A dpi formulation comprises a solvate of beclomethasone and is substantially free of excipient and free of carrier. The solvate particles are of size 0.5 to 10 microns and are obtained by crystallization of the steroid in the presence of ultrasound.

### FIELD

The present invention relates to dry powder inhaler formulations of steroids, in particular dry powder inhaler formulations of beclomethasone.

### BACKGROUND

Delivery of steroids to the lungs via aerosol is widely known and used for the treatment of a number of diseases, including asthma, airways disease and chronic obstructive pulmonary disease (COPD). Formulations are generally administered via dry powder inhaler (dpi), metered dose inhaler (mdi) and, to a lesser extent, nebuliser.

### PROBLEMS

A dpi formulation of beclomethasone is known, containing beclomethasone diproprionate particles falling in the size range 2-5 microns in combination with lactose particles with a recommended particle size of 60-90 microns. This formulation is successfully used for administration of beclomethasone, with apparently suitable particle size and content uniformity data. However, to obtain satisfactory content uniformity data the lactose and active have to be mixed very carefully. Additionally, according to the National Digestive Diseases Information Clearing House, an estimated 30 to 50 million Americans (about 25% of the United States population) are lactose intolerant and can thus experience adverse reactions to formulations comprising lactose.

A further disadvantage of these formulations is that the effective dose of beclomethasone at point of delivery to the patient is rather lower than that contained in the formulation, it being acknowledged that a certain loss of product occurs during delivery. This loss is compensated for in the amount of active included in the formulation, a solution regarded as acceptable. Nevertheless, it would be desirable to reduce this loss: any loss is to some degree uncontrollable and hence affects the reliability of dosing.

A method of preparing small crystals is described in WO 02/089942, in which crystallization occurs in the presence of ultrasound. WO 2004/073827 describes preparation of aerosol formulations for mdi and dpi uses, again using ultrasound during crystallization of the active component. WO 2010/007447 describes a process for increasing the crystallinity of a solid material and describes the use of the process for preparing particles for dpi formulations.

An aim of the present invention is to provide alternative, preferably improved beclomethasone dpi formulations and methods of making the same.

### INVENTION

Accordingly, the invention provides a dpi formulation, substantially consisting of a solvate of beclomethasone. The beclomethasone solvate is preferably obtained by crystallisation in the presence of ultrasound.

In an embodiment of the invention, the beclomethasone solvate is obtained by forming a suspension of (i) droplets containing beclomethasone dissolved in a solvent, in (ii) a non-solvent of beclomethasone, and applying ultrasound to the droplets to form crystallised beclomethasone particles.

In an embodiment of the invention the beclomethasone solvate is obtained by drying a solution of beclomethasone in a solvent to obtain solid, preferably substantially amorphous particles of beclomethasone, which are then contacted with a non-solvent of beclomethasone and subjected to ultrasound to form crystallized beclomethasone particles. The solution may be in the form of droplets. The drying may be carried out by rapid precipitation, freeze drying, lyophilisation, rapid expansion of supercritical solution, spray drying or mixtures thereof.

The invention thus enables preparation of such formulations substantially without carriers or excipients, avoiding problems associated with carrier intolerance and avoiding the need for provision of content uniformity data.

Suitable non-solvents for use in formation of the solvate include water and C₅-C₇ hydrocarbons. The C₅-C₇ hydrocarbon is typically non-cyclic, straight or branched and in a preferred embodiment of the invention the C₅-C₇ hydrocarbon is heptane. In a specific embodiment the heptane is n-heptane.

Preferred formulations of the invention comprise beclomethasone solvate particles of diameter 0.5-10 microns, more preferably 0.5-5 microns. Further, it is preferred that a substantial proportion of the product be within these stated size ranges so that a substantial proportion will reach the patient's lungs, and preferably at least 75% (by number), more preferably at least 90% (by number) of the solvate particles are within the stated size range. It is further preferred that the solvate particle size distribution is within the range X₁₀=>0.1 microns, X₅₀=<5 microns and X₉₀=<10 microns, more preferably within the range X₁₀=>0.5 microns, X₅₀=<3 microns and X₉₀=<7 microns and in a particular embodiment of the invention the particle size distribution is within the range X₁₀=>0.5 microns, X₅₀=<3 microns and X₉₀=<5 microns.

Particle size or particle diameter as used herein can be suitably determined by laser diffraction based methods, for example as described in ISO Standard 13320-1. Laser diffraction particle sizing apparatus such as the Malvern Mastersizer 2000™ can be used.

In use of formulations of the invention typically at least 80% (by weight) of the dose of active is delivered to the patient, preferably at least 85% (by weight) and more preferably at least 90% (by weight) of the dose of active is delivered to the patient. In a particular embodiment of the invention at least 95% (by weight) of the dose of active is delivered to the patient and in a further embodiment of the invention at least 97% (by weight) can be delivered to the patient. In a yet further embodiment of the invention up to 98% (by weight) of the dose of active is delivered to the patient. In alternative embodiments of the invention up to 99% (by weight) or up to 100% (by weight) of the dose of active can be delivered to the patient.

In use of the invention beclomethasone solvate particles are obtained having a morphology that is visibly homogenous by SEM imaging, and are characterized by substantially smooth surfaces and rounded edges.

In formulations of the invention beclomethasone can comprise 90% or more of the formulation by weight and carrier or excipient can comprise up to 10% of the formulation by weight. It is preferred that 95% or more of the formulation by weight is beclomethasone and up to 5% of the formulation by weight is carrier or excipient. In a particularly preferred embodiment of the invention 98% or more of the formulation by weight is beclomethasone and up to 2% of the formulation by weight is carrier or excipient. In a further embodiment beclomethasone comprises at least 99% (by weight) of the formulation. In a particular embodiment of the invention beclomethasone comprises 100% (by weight) of the formulation.

Typically dpi formulations of the invention consist essentially of beclomethasone and preferred formulations are substantially free of excipient and free of carrier. Preferred formulations of the invention substantially comprise beclomethasone and any additional components are present in de minimis amounts.

Suitable dpi formulations of the invention can comprise individual doses of 15 to 300 mcg of beclomethasone; preferably the individual doses are within the range 20 to 200 mcg. A preferred formulation of the invention comprises individual doses of about 25 mcg of beclomethasone, in another embodiment of the invention the formulations comprise individual doses of about 50 mcg of beclomethasone and in a yet further embodiment of the invention the formulations comprise individual doses of about 125 mcg of beclomethasone. Alternatively, dpi formulations of the invention can comprise individual doses of about 37.5 mcg of beclomethasone, in another embodiment of the invention the formulations comprise individual doses of about 75 mcg of beclomethasone and in a yet further embodiment of the invention the formulations comprise individual doses of about 187.5 mcg of beclomethasone.

The present invention also provides a method of preparing a dpi formulation of a solvate of beclomethasone comprising (i) crystallising beclomethasone solvate particles in the presence of ultrasound, and (ii) formulating the crystallised particles into a DPI formulation.

In an embodiment of the invention, the method comprises forming a suspension of (i) droplets containing beclomethasone dissolved in a solvent, in (ii) a non-solvent of beclomethasone, and applying ultrasound to the droplets to form crystallised beclomethasone solvate particles.

Beclomethasone is suitably crystallized by forming a solution of steroid in a solvent, forming a suspension of droplets of the solution in a non-solvent of the steroid, and applying ultrasound to the droplets. The steroid in the suspended droplets, which may be mainly or entirely beclomethasone, crystallizes to form particles of a generally spherical type. More specifically, it is crystallized by dissolving it in a solvent, forming droplets of the solution, for example by generating an aerosol from this solution, forming a dispersion of the droplets in a non-solvent of the steroid and subjecting the droplets to ultrasound to initiate or effect crystallization of the steroid.

In an alternative embodiment of the invention, the method comprises drying a solution of beclomethasone in a solvent to obtain solid, preferably substantially amorphous particles, which are then contacted with a non-solvent of beclomethasone and subjected to ultrasound to form crystallized beclomethasone particles. The solution may be in the form of droplets. The drying may be carried out by rapid precipitation, freeze drying, lyophilisation, rapid expansion of supercritical solution, spray drying or mixtures thereof.

Droplets can be prepared by electrohydrodynamic spraying, atomizing using high pressure, spray nozzles, nebulisers, transducers such as piezoelectric transducers or ultrasonic transducers or other aerosol generators.

To obtain the desired particle size of the crystalline steroid solvate the size of the droplets and the amount of steroid in the solvent are varied and controlled. The process is to a certain extent empirical as different systems operating under similar conditions will produce different end particle sizes. However, the droplets should generally be micron sized, say in the range 1-100 microns, preferably 3-30 microns to yield crystals in the size range 0.5-10 microns.

To obtain more generally spherical crystals it is preferred that the droplets of solvent contain a high proportion of steroid. Solvent evaporates from the solvent droplets in the aerosol and this can be controlled and optimized so that the droplets when they are collected in or combined with the beclomethasone non-solvent contain at least 80%, more preferably at least 90%, more preferably at least 95% steroid by weight of droplet.

In embodiments of the invention in which droplets are subjected to drying prior to contact with the non-solvent, the dried particles should generally be micron sized, say in the range up to 10 microns, preferably 0.1-10 microns to yield crystals in the size range 0.5-10 microns. Manipulation of the drying conditions and subsequent ultrasound treatment allows crystals to be formed having predetermined characteristics. Such characteristics may include particle morphology, surface free energy, particle size distribution, desired polymorph and, in terms of isolated particles, flowability, reduced electrostatic and cohesive/adhesive properties.

Hence by variation of a number of parameters, including % product in the droplets and droplet size, the ultimate crystal particle size can be controlled so that particles within the ranges 0.5-10 microns, preferably 0.5-5 microns are obtained. It is further preferred that the solvate particle size distribution is within the range X₁₀=>0.1 microns, X₅₀=<5 microns and X₉₀=<10 microns, more preferably within the range X₁₀=>0.5 microns, X₅₀=<3 microns and X₉₀=<7 microns and in a particular embodiment of the invention the particle size distribution is within the range X₁₀=>0.5 microns, X₅₀=<3 microns and X₉₀=<5 microns.

Suitable solvents for beclomethasone are alcohols and ketones, in particular low molecular weight ketones, alcohols and halogenated alkanes, specific examples being acetone, ethanol, methanol and dichloromethane. In a preferred embodiment of the invention the solvent is or comprises methanol.

The non-solvent should dissolve a very low amount of the steroid, preferably not more than 0.1% w/w; it may be miscible with the solvent and an emulsifier or other agent may be added to aid stability of the droplets suspension. Suitable non-solvents include C₅-C₇ hydrocarbons that can be non-cyclic, straight or branched. A preferred non-solvent is heptane and in a specific embodiment of the invention the heptane is n-heptane. A further preferred non-solvent for use in the method of the invention is water.

Crystallization is effected or initiated by applying ultrasound to the steroid. Crystallization is also effected or initiated by applying ultrasound to the solvate. The ultrasound may be applied continuously or discontinuously such as in a pulsed manner. It may be applied using a variety or devices, such as a probe inserted into the suspension.

Whilst the frequency and amplitude may vary, beclomethasone may be crystallized in the presence of ultrasound having frequency from 20 kHz to 5 MHz.

Separately, ultrasound may have an intensity of 0.2 W/cm² or higher, or 0.3 W/cm² or higher.

In embodiments of the invention an ultrasound frequency of 16 kHz to 1 MHz can be used.

The method of the invention typically further comprises drying the solvate particles. Suitable drying methods include spray drying and drying by super-critical CO₂. In a preferred embodiment of the invention the particles are dried by spray drying.

A specific embodiment of the invention provides a method of preparing a dpi formulation of beclomethasone, comprising:—
(a) forming a suspension of (i) droplets containing beclomethasone dissolved in a solvent, in (ii) n-heptane or water,
(b) applying ultrasound to the droplets to form crystallised beclomethasone particles of 0.5-5 microns,
(c) drying the particles by spray drying and
(d) packaging the dried particles into an excipient free dpi dosage form suitable for administration to patients.

A further specific embodiment of the invention provides a method of preparing a dpi formulation of beclomethasone, comprising:—
(a) forming a solution of beclomethasone in a solvent,
(b) subjecting the solution to a process selected from the group consisting of rapid precipitation, freeze drying, lyophilisation, rapid expansion of supercritical solution, spray drying or mixtures thereof, wherein the beclomethasone is converted into substantially dry solid material,
(c) optionally isolating the beclomethasone from the liquid or gaseous components of the process of step (b),
(d) treating the beclomethasone from step (b) or (c), with n-heptane or water,
(e) applying ultrasound to the beclomethasone when it is in contact with the n-heptane or water of step (d) to form crystallised beclomethasone particles of 0.5-5 microns,
(f) drying the particles by spray drying, and
(g) packaging the dried particles into an excipient free dpi dosage form suitable for administration to patients.

In a further specific embodiment of the method the solvent comprises methanol.

A yet further specific embodiment of the invention provides a method of preparing a dpi formulation of beclomethasone, comprising:—
(a) forming a suspension of (i) droplets containing beclomethasone dissolved in a solvent, in (ii) n-heptane or water,
(b) applying ultrasound to the droplets to form crystallised beclomethasone having a particle size distribution within the range X₁₀=>0.5 microns, X₅₀=<3 microns and X₉₀=<5 microns,
(c) drying the particles by spray drying and packaging the dried particles into an excipient-free dpi dosage form suitable for administration to patients.

A still further specific embodiment of the invention provides a method of preparing a dpi formulation of beclomethasone, comprising:—
(a) forming a solution of beclomethasone in a solvent,
(b) subjecting the solution to a process selected from the group consisting of rapid precipitation, freeze drying, lyophilisation, rapid expansion of supercritical solution, spray drying or mixtures thereof, wherein the beclomethasone is converted into substantially dry solid material,
(c) optionally isolating the beclomethasone from the liquid or gaseous components of the process of step (b),
(d) treating the beclomethasone from step (b) or (c), with n-heptane or water,
(e) applying ultrasound to the beclomethasone when it is in contact with the n-heptane or water of step (d) to form crystallised beclomethasone having a particle size distribution within the range X₁₀=>0.5 microns, X₅₀=<3 microns and X₉₀=<5 microns,
(f) drying the particles by spray drying, and
(g) packaging the dried particles into an excipient free dpi dosage form suitable for administration to patients.

Reference herein to beclomethasone is reference to the drug substance in any of its suitable and available forms, including salts and other derivatives thereof, such as but not limited to beclomethasone dipropionate and beclomethasone valerate, etc.

### EXAMPLES

### Example 1

Beclomethasone was crystallized utilizing ultrasound. Briefly, this method comprised formation of a drug substance solution followed by its atomization, controlled evaporation of the solvent by spray drying, resulting in substantially amorphous particles, which were then contacted with a non-solvent of beclomethasone and subjected to ultrasound to form a product slurry comprising crystallized beclomethasone particles. The product slurry was then transferred to solid isolation, by spray-drying or supercritical carbon dioxide drying. The method was carried out by Prosonix Ltd of Oxford, UK and further details of this method are as described in WO 2010/007447.

Beclomethasone hydrate obtained by crystallization in the presence of ultrasound

### Protocol:

Input: 6 g of anhydrous beclomethasone diproprionate (BDP)
3% w/v solution of anhydrous BDP in methanol was atomized, spray dried and sonoprocessed in water
Temperature: 0° C.
Particles were isolated by spray drying

Differential scanning calorimetry (DSC) and TGA following isolation by spray drying showed highly crystalline BDP hydrate.

SEM showed particles with smooth surfaces and homogeneous morphology. Dry Sympatec PSD analysis confirmed that the particle size distribution was well within the inhalation range.

Table 1 shows the results of dry Sympatec PSD analysis:

| Cumulative distribution Q3 (%) | Particle Size (μ) |
|---|---|
| X₁₀ | 0.51 |
| X₅₀ | 1.35 |
| X₉₀ | 3.17 |

In order to evaluate the effect of humidity on prolonged storage processed BDP hydrate was subjected to 20% relative humidity (RH) for 48 hours.

DVS mass plot of the processed BDP hydrate showed that during storage the sample initially underwent considerable weight loss due to partial dehydration. The sample achieved a steady state after about 1500 minutes. The loss of water from the sample is likely to reflect the loss of free water remaining in the sample after spray drying, as this drying technique is usually not 100% efficient.

These results indicate that BDP formed a hydrate at a very low moisture content, and is anticipated to retain stability on prolonged storage.

The sample recovered after storage was analysed by DSC, TGA, PSD and SEM.

The DSC trace of the stored sample indicated no variation in the thermal behaviour of the sample post-humidity treatment. The hydrated sample exhibited higher stability on prolonged storage than anhydrous BDP.

PSD showed no significant variation of particle size and SEM analysis showed identical morphology to the pre-storage sample.

Table 2 shows the results of dry Sympatec PSD analysis of the post-storage sample:

| Cumulative distribution Q3 (%) | Particle Size (μ) |
|---|---|
| X₁₀ | 0.51 |
| X₅₀ | 1.37 |
| X₉₀ | 2.95 |

### Example 2

Beclomethasone is crystallized utilizing ultrasound. Briefly, this method comprises formation of a drug substance solution followed by its atomization, controlled evaporation of the solvent, collection of the pre-concentrated viscous droplets in a vessel containing non-solvent and crystallisation via nucleation with power ultrasound. The product slurry is then transferred to solid isolation, by spray-drying or supercritical carbon dioxide drying. Further details of this method are as described in WO 2004/073827.

Beclomethasone hydrate obtained by crystallization in the presence of ultrasound

### Protocol:

Input: 6 g of anhydrous beclomethasone diproprionate (BDP)
3% w/v solution of anhydrous BDP in methanol is atomized and sonoprocessed in water
Temperature: 0° C.
Particles are isolated by spray drying

### Example 3

Crystallization in the presence of ultrasound (as per Example 1) with n-heptane

### Protocol:

Input: 6 g of anhydrous beclomethasone diproprionate (BDP)
3% w/v solution of anhydrous BDP in methanol was atomized, spray dried and sonoprocessed in n-heptane
Temperature: 0° C.
Particles were isolated by spray drying

Differential scanning calorimetry (DSC) and TGA following isolation by spray drying confirmed that the isolated material was an n-heptane solvate and highly crystalline.

SEM showed very homogeneous particles with smooth surfaces and well defined pebble-like morphology. Dry Sympatec PSD analysis confirmed that the particle size distribution was extremely promising and within the inhalation range.

Table 3 shows the results of dry Sympatec PSD analysis:

| Cumulative distribution Q3 (%) | Particle Size (μ) |
|---|---|
| X₁₀ | 0.69 |
| X₅₀ | 2.41 |
| X₉₀ | 4.67 |

In order to evaluate the effect of humidity on prolonged storage, processed BDP heptane was subjected to 20% relative humidity (RH) for 48 hours.

DVS mass plot of the processed BDP heptane showed that the material maintained full stability in terms of change of mass.

The sample recovered after storage was analysed by DSC, TGA, PSD and SEM.

The DSC trace of the stored sample indicated no variation in the thermal behaviour of the sample post-humidity treatment.

PSD showed no significant variation of particle size and SEM analysis showed identical morphology to the pre-storage sample.

Table 4 shows the results of dry Sympatec PSD analysis of the post-storage sample:

| Cumulative distribution Q3 (%) | Particle Size (μ) |
|---|---|
| X₁₀ | 0.68 |
| X₅₀ | 2.44 |
| X₉₀ | 5.29 |

### Example 4

Crystallization in the presence of ultrasound (as per Example 2) with n-heptane

### Protocol:

Input: 6 g of anhydrous beclomethasone diproprionate (BDP)
3% w/v solution of anhydrous BDP in methanol is atomized and sonoprocessed in n-heptane
Temperature: 0° C.
Particles are isolated by spray drying

### Example 5

### Beclomethasone Formulation

A beclomethasone DPI formulation is prepared, by dissolving beclomethasone in a solvent and then forming a suspension of the beclomethasone solution in a non-solvent, and crystallizing the beclomethasone by application of ultrasound, as described in WO 2004/073827.

The operating parameters including flow rate and ultrasound power are varied so as to obtain a particle size for crystallized beclomethasone substantially within the size range 2-3 microns.

The beclomethasone solvate obtained is subjected to end-sterilization by irradiation to yield end DPI formulations to be dispensed in the following individual doses:—

| | | | |
|---|---|---|---|
| 1 | Beclomethasone | 25 | mcg |
| 2 | Beclomethasone | 50 | mcg |
| 3 | Beclomethasone | 125 | mcg |
| 4 | Beclomethasone | 37.5 | mcg |
| 5 | Beclomethasone | 75 | mcg |
| 6 | Beclomethasone | 187.5 | mcg |

### Example 6

### Beclomethasone Formulation

A beclomethasone DPI formulation is prepared, by dissolving beclomethasone in a solvent and evaporating the solvent by spray drying under controlled conditions, resulting in substantially amorphous particles, which are then contacted with a non-solvent of beclomethasone, and crystallizing the beclomethasone by application of ultrasound, as described in WO 2010/007447.

The operating parameters including flow rate and ultrasound power are varied so as to obtain a particle size for crystallized beclomethasone substantially within the size range 2-3 microns.

The beclomethasone solvate obtained is subjected to end-sterilization by irradiation to yield end DPI formulations to be dispensed in the following individual doses:—

| | | | |
|---|---|---|---|
| 1 | Beclomethasone | 25 | mcg |
| 2 | Beclomethasone | 50 | mcg |
| 3 | Beclomethasone | 125 | mcg |
| 4 | Beclomethasone | 37.5 | mcg |
| 5 | Beclomethasone | 75 | mcg |
| 6 | Beclomethasone | 187.5 | mcg |

The invention thus provides beclomethasone-containing dpi formulations and methods for the manufacture thereof.

1-30. (canceled)

31. A Dry Powder Inhaler (DPI) formulation comprising 90% or more of the formulation by weight of a solvate of beclomethasone and up to 10% of the formulation by weight of a carrier or excipient and, wherein the beclomethasone solvate is obtained by forming a suspension of (i) droplets containing beclomethasone dissolved in a solvent, in (ii) water of a C₅-C₇ hydrocarbon non-solvent of beclomethasone, and applying ultrasound to the droplets to form crystallised beclomethasone particles.

32. A DPI formulation according to claim 31, wherein the C₅-C₇ hydrocarbon is non-cyclic, straight or branched.

33. A DPI formulation according to claim 32, wherein the C₅-C₇ hydrocarbon is heptane.

34. A DPI formulation according to claim 33, wherein the heptane is n-heptane.

35. A DPI formulation according to claim 31, comprising beclomethasone solvate particles of diameter 0.5-10 microns.

36. A DPI formulation according to claim 35, wherein the particle size distribution is within the range X₁₀≧0.1 microns, X₅₀≦5 microns and X₉₀≦10 microns.

37. A DPI formulation according to claim 36, wherein the particle size distribution is within the range X₁₀≧0.5 microns, X₅₀≦3 microns and X₉₀≦7 microns.

38. A DPI formulation according to claim 37, wherein the particle size distribution is within the range X₁₀≧0.5 microns, X₅₀≦3 microns and X₉₀≦5 microns.

39. A DPI formulation according to claim 31, comprising beclomethasone particles having a morphology that is visibly homogenous by SEM imaging, with substantially smooth surfaces and rounded edges.

40. A DPI formulation according to claim 31, wherein 95% or more of the formulation by weight is beclomethasone and up to 5% of the formulation by weight is carrier or excipient.

41. A DPI formulation according to claim 40, wherein 98% or more of the formulation by weight is beclomethasone and up to 2% of the formulation by weight is carrier or excipient.

42. A DPI formulation according to claim 31, wherein the formulation consists essentially of beclomethasone.

43. A method of preparing a DPI formulation of a solvate of beclomethasone according to claim 31, comprising (i) crystallizing beclomethasone solvate particles in the presence of ultrasound, and (ii) formulating the crystallised particles into a DPI formulation.

44. A method according to claim 43, wherein step 1 comprises forming a suspension of (i) droplets containing beclomethasone dissolved in a solvent, in (ii) water of a C₅-C₇ hydrocarbon non-solvent of beclomethasone, and applying ultrasound to the droplets to form crystallised beclomethasone solvate particles.

45. A method according to claim 44, wherein the C₅-C₇ hydrocarbon is non-cyclic, straight or branched.

46. A method according to claim 45, wherein the C₅-C₇ hydrocarbon is heptane.

47. A method according to claim 46, wherein the heptane is n-heptane.

48. A method according to claim 44, further comprising drying the crystallised steroid particles.

49. A method according to claim 48, wherein the particles are dried by spray drying.

50. A method of preparing a DPI formulation of beclomethasone, comprising:
(a) forming a suspension of (i) droplets containing beclomethasone dissolved in a solvent, in (ii) n-heptane or water,
(b) applying ultrasound to the droplets to form crystallised beclomethasone particles of 0.5-microns,
(c) drying the particles by spray drying and
(d) packaging the dried particles into an excipient free DPI dosage form suitable for administration to patients.

51. A method according to claim 50, wherein the solvent comprises methanol.

52. A method of preparing a DPI formulation of beclomethasone, comprising:
(a) forming a suspension of (i) droplets containing beclomethasone dissolved in a solvent, in (ii) n-heptane or water,
(b) applying ultrasound to the droplets to form crystallised beclomethasone having a particle size distribution within the range of X₁₀≧0.5 microns, X₅₀≦3 microns and X₉₀≦5 microns,
(c) drying the particles by spray drying and
(d) packaging the dried particles into an excipient free DPI dosage form suitable for administration to patients.
